(19) United States
(12) Patent Application Publication
(10) Pub. No.: US 2024/0287556 A1
(43) Pub. Date: Aug. 29, 2024

(54) USING BIOSOURCED RAWS FROM WASTE STREAMS TO MAKE SOURCE MATERIALS FOR OILFIELD CHEMICAL MANUFACTURE

(71) Applicant: Halliburton Energy Services, Inc., Houston, TX (US)

(72) Inventors: Ronald Bosch, Houston, TX (US); Antonio Recio, III, Houston, TX (US); Enrique Antonio Reyes, Houston, TX (US)

(73) Assignee: Halliburton Energy Services, Inc., Houston, TX (US)

(21) Appl. No.: 18/115,585

(22) Filed: Feb. 28, 2023

(51) Int. Cl. C12P 7/6436; C12N 1/20; C12N 9/10; C12P 7/6409

(52) U.S. Cl. CPC C12P 7/6436; C12N 1/20; C12N 9/1029; C12P 7/6409; C12Y 203/01075

(57) ABSTRACT

A method may include: introducing a hydrocarbon containing waste source into a bioreactor, metabolizing at least a portion of hydrocarbons from the hydrocarbon containing waste source using a bacterial organism to form a biologically derived intermediate product; and introducing at least a portion of the biologically derived intermediate product into a chemical reactor and reacting at least a portion of the biologically derived intermediate product to form a chemical product.

### BACKGROUND

In industry, chemicals are utilized to exert a specific engineered effect. Chemicals may include several broad classes of chemicals such as wetting agents, corrosion inhibitors, water clarifiers, stimulation chemicals, sand consolidation chemicals, ionic liquids, and petrochemical treatment chemicals, for example. The functionality of the chemicals is typically engineered into the chemical during production of the chemical. For example, a chemical may be engineered to have desired functional groups, carbon chain lengths, degree of isomerization, or any other features to exert a desired chemical or physical property. chemicals are typically produced using petroleum hydrocarbons as a raw material. While there are some biological sources and biological methods for producing chemicals, the biologically-sources chemicals are often of low efficacy and/or quality.

### BRIEF DESCRIPTION OF THE DRAWINGS

These drawings illustrate certain aspects of some of the embodiments of the present disclosure and should not be used to limit or define the disclosure.

FIG. 1 is a block flow diagram which illustrates a process to use biologically derived raw materials for production of chemicals in accordance with some embodiments of the present disclosure.

FIG. 2 is a schematic illustration of a bioreactor in accordance with some embodiments of the present disclosure.

FIG. 3 is a schematic illustration of a process for producing a chemical from a hydrocarbon containing waste source in accordance with some embodiments of the present disclosure.

FIG. 4 is a schematic illustration of a process for producing a chemical from a hydrocarbon containing waste source in accordance with some embodiments of the present disclosure.

### DETAILED DESCRIPTION

The present disclosure may generally relate to production of chemicals. In particular, the disclosure may relate to a process including providing a hydrocarbon containing waste source, using the hydrocarbon containing waste source as a feedstock to a bioreactor to produce a biologically derived intermediate product, and using the biologically derived intermediate product to produce a chemical. chemicals which may be produced by the process include, but are not limited to, wetting agents, corrosion inhibitors, water clarifiers, stimulation chemicals, sand consolidation chemicals, salt control chemicals, ionic liquids, and petrochemical treatment chemicals, for example.

One class of biologically derived intermediate products which may be prepared according to the methods and processes disclosed herein are wax esters such as by acyl-CoA:diacylglycerol acyltranserface (WS/DGAT; EC 2.3.1.75) which is an enzyme that catalyzes the esterification of fatty acyl coenzyme A and a fatty alcohol.

Wax esters and triacylglycerols as raw materials have found use in various consumer goods, such as lubricants, cosmetics, paints, perfumes, candles, sealants, inks, and polishes. It is well known that wax esters and triacylglycerols can be harvested form several eukaryotic organisms including plants, fungi, and animals. Prokaryotic organisms such as Mycobaterium, Rhodococcus, Nocardia, and Streptomyces with the correct metabolic inputs such as a hydrocarbon as described herein, can be engineered to produce a selected biologically derived intermediate product. For example, the above referenced prokaryotic organisms may have >20% of biomass accumulate as lipids including as fats, waxes, sterols, monoglycerides, diglycerides, and phospholipids, for example.

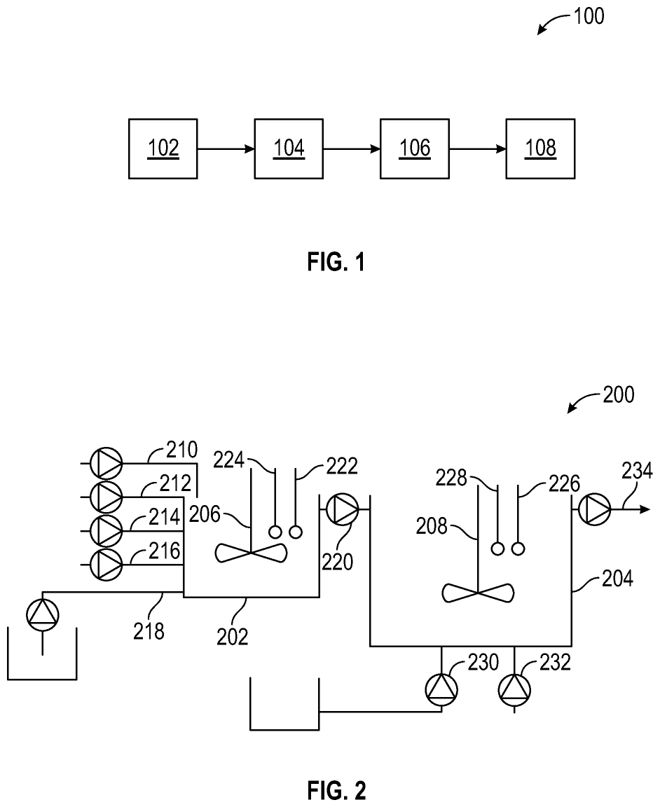

FIG. 1 is a block flow diagram of a process 100 for producing chemicals. Process 100 begins at block 102 where a hydrocarbon containing waste source is provided. Hydrocarbon containing waste source can be any source which contains a hydrocarbon which can be used in a metabolic process by a bacterial organism. Some non-limiting waste sources may include industrial sources such as wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, and mine waste from tar sands mining as well as produced hydrocarbons such as crude oil, gas, and asphaltenes.

One advantage to using hydrocarbon containing waste sources is that biologically produced raw materials can be sourced nearer to the wastewater production, and thus closer to the use points. Further, using waste sources may allow for processes which can be run at lower pressures and temperatures than petrochemicals processes, thus requiring much less infrastructure and energy costs. Additionally, biologically produced raw materials will reduce carbon footprint in significant amounts due to reusing carbon lost to wastewater streams to provide a raw materials stream to plants that currently use petrochemicals to produce chemical. Biologically produced raw materials may also shorten supply chains to chemical production plants due to these plants often being necessarily far from their petrochemical production sources which may reduce transportation burden and the associate costs. Additionally, biological pathways to production of the raw materials can be used to make products that are not readily available at the plant location, and thus be used to increase in country value in places where there is benefit to producing locally.

One suitable source of hydrocarbon containing waste is produced water from oil and gas wells. Produced water contains different organic and inorganic solutes which can be used as feedstock for the bioreactor. In some examples, the amount of water can be reduced to facilitate the biosynthesis of reaction products, and if necessary, strip certain contaminants that can hinder the biosynthetic process such as H₂S for example.

From block 102, process 100 proceeds to block 104, a bioreactor, where the hydrocarbon containing source from block 102 is utilized as a feed to a bioreactor 104 comprising at least one bacterial organism. The hydrocarbon containing waste source may include at least one type of hydrocarbon in sufficient quantity to enable the bacterial organism in the bioreactor to metabolize the hydrocarbon to form a biologically derived intermediate product. Some non-limiting hydrocarbons may include aliphatic hydrocarbons, aromatic hydrocarbons, sugars, fats, organic acids such as carboxylic acids, alkyl and/or aryl sulfates, alkyl or aryl sulfides, C-10 to C-28 Fatty Esters, C-10 to C-28 partially and fully saturated fats, C-10 to C-28 unsaturated fats, C-10 to C-28 saturated fatty acids, C-10 to C-28 unsaturated fatty acids, proteins, amino acids, linear and branched alcohols including fusel alcohols and fusel oil, simple and complex sugars, starches, cellulose, and combinations thereof.

The bioreactor 104 may include any suitable type of bioreactor, including, but not limited to, batch bioreactors, continuously stirred bioreactors, bubble column bioreactors, airlift bioreactors, fluidized bed bioreactors, packed bed bioreactors, photo bioreactors, and/or membrane bioreactors for example. The bioreactor may include further supporting equipment such as heaters, coolers, pumps, stirring implements, light source, as well as inputs including, but not limited to, oxygen, air, carbon dioxide, water, nitrogen source such as NH₄Cl, phosphorous source such as Na₂HPO₄, and nutrients, for example. The desired biologically derived intermediate product may inform the choice of bioreactor, bacterial organism, and inputs needed to the bioreactor.

The bacterial organism in the bioreactor 104 may be selected based at least in part on the identity of the hydrocarbon in the hydrocarbon containing waste source and an ability of the bacterial organism to metabolize the hydrocarbon into a biologically derived intermediate product. In some embodiments, the bacterial organism may be engineered or selected to have a desired functionality to metabolize the hydrocarbon into a biologically derived intermediate product. In some embodiments, the bacterial organism may be genetically modified to include a gene which targets the production of a particular biologically derived intermediate product from a hydrocarbon. In further embodiments the bacterial organism may be serially selected to target a production of a biologically derived intermediate product.

The biologically derived intermediate product may include any chemical that can be used as a feedstock to make a chemical. Some non-limiting biologically derived intermediate products may include, without limitation, alcohols such as methanol, ethanol, butanol, hexanol, or in general, C₁-C₃₀ alcohols, C₁-C₃₀ hydrocarbons, C₁-C₃₀ alkanes, C₁-C₃₀ alkenes, C₁-C₃₀ alkynes, C₆-C₃₀ aromatics, C₂-C₃₀ acetates, C₂-C₃₀ alkaline earth acetates, carboxylic acids such as acetic acid, or in general, C₂-C₃₀ carboxylic acids, C₂-C₃₀ olefins, syngas (mixture of hydrogen and carbon monoxide), C₂-C₃₀ wax esters, triacylglycerols with C₂-C₃₀ fatty acid side chains, C₂-C₃₀ aldehydes, C₂-C₃₀ ketones, C₂-C₃₀ acid anhydrides, C₂-C₃₀ acyl halides, C₂-C₃₀ ethers, C₂-C₃₀ epoxies, C₂-C₃₀ mono and higher order amines, C₂-C₃₀ amides, C₂-C₃₀ enamines, C₂-C₃₀ ketals, C₂-C₃₀ lactones, C₂-C₃₀ nitrates, C₂-C₃₀ nitrites, C₂-C₃₀ nitriles, C₂-C₃₀ nitro containing compounds, C₂-C₃₀ alkanolamides, C₂-C₃₀ amidoamines, C₂-C₃₀ pyrenes, C₂-C₃₀ nitroso containing compounds, C₂-C₃₀ imines, C₂-C₃₀ imides, C₂-C₃₀ azides, C₂-C₃₀ cyanates, C₂-C₃₀ isocyanates, C₂-C₃₀ azo compounds, C₂-C₃₀ thiols, C₂-C₃₀ sulfides, C₂-C₃₀ disulfides, C₂-C₃₀ sulfoxides, C₂-C₃₀ sulfones, C₂-C₃₀ sulfinic acids, C₂-C₃₀ sulfonic acids, C₂-C₃₀ sulfonate esters, C₂-C₃₀ tyiocyanate, C₂-C₃₀ iscothiocyanate, C₂-C₃₀ thials, C₂-C₃₀ thioketones, starches, and aldehydes, and combinations thereof. In embodiments, a biologically derived intermediate product may include a combination of functional groups. Some particular biologically derived intermediate products may include, without limitation, C₂-C₃₀ wax esters, C₂-C₃₀ triacylglycerols, and polyhydroxyalkanoates. In embodiments, the biologically derived intermediate product may have a biogenic carbon content of greater than 50 wt. % as measured by ASTM D6866-22. Alternatively, the biologically derived intermediate product may have a biogenic carbon content of greater than 75 wt. %, greater than 90 wt. %, or greater than 99 wt. %.

The bacterial organism may contain one or more enzymes which catalyze the metabolism of the hydrocarbon to the one or more intermediate products which may include one or more functional groups. A bacterial organism may be screened to select for particular functionality of the enzymes based on a desired biologically derived intermediate product. Some suitable bacterial organisms may include, without limitation, Acidobacteria bacterium, Acidothermus cellulolyticus, Acinetobacter baumannii, Acinetobacter baylyi, Rhodoccus opacus, Acinetobacter baumannii, Aeromonas hydrophila, Aeromonas salmonicida, Alcaligenes europhus, Alcanivorax borkumensis, Alcanivorax jadensis, Alteromonas macleodii, Anaeromyxobacter dehalogenans, Anaeromyxobacter, Rhodoccus erythroplis, Arabidopsis thaliana, Bradyrhizobium japonicum, Cryptococcus curvatus, Erythrobacter litoralis, Fundibacter jadensis, gamma proteobacterium, Hahella chejuensis, Rhodoccus fascians, marine gamma proteobacterium, Marinobacter algicola, Marinobacter aquaeolei, Marinobacter hydrocarbinoclasticus, Rhodoccus ruber, Rhodoccus jostii; Nocardia asteroides, Nocardia corallina, Nocardia globerula, Nocardia resticta; Methylibium petroleiphilum, Microscilla marina, Mortierella alpina, Mus musculus, Mycobacterium abscessus, Mycobacterium avium, Mycobacterium avium, Mycobacterium bovis, Mycobacterium gilvum, Mycobacterium leprae, Mycobacterium marinum, Mycobacterium smegmatis, Mycobacterium tuberculosis, Mycobaterium smegmatis, Mycobacterium ratisbonense; Mycobacterium ulcerans, Mycobacterium vanbaalenii, Myxococcus xanthus, Natronomonas pharaonis, Nocardia farcinica, Dietzia maris; Gordonia amarae, Streptomyces coelicolor, Photobacterium profundum, Plesiocystis pacifica, Polaromonas naphthalenivorans, Psudomonas aeruginosa, Psychrobacter arcticus, Psychrobacter cryohalolentis, Rhodococcus opacus, Rhodoferax ferrireducens, Rhodoferax ferrireducens, Roseiflexus sp., Roseiflexus castenholzii, Saccharomyces cerevisiae, Saccharopolyspora erythraea, Salinibacter ruber, Simmodsia chinensis, Solibacter usitatus, Sphingopyxis alaskensis, Stigmatella aurantiaca, Streptomyces avermitilis, Streptomyces coelicolor, Streptomyces griseus, Ustilago maydis, Desulfovibrio, Pseudomonas, Azotobacter, Escheri coli, and combinations thereof.

From block 104 in FIG. 1, process 100 proceeds to block 106 where the effluent from the bioreactor containing the biologically derived intermediate product from block 104 is utilized as a feed to a separation unit 106. The separation unit 106 may include any suitable unit operations such as extraction, leaching, flotation, flocculation, filtration, fractional distillation, precipitation, recrystallization, stripping, chelation, filtration, centrifugation, or any combinations thereof, to separate at least a portion of the biologically derived intermediate product from the bioreactor effluent to produce a purified biologically derived intermediate product suitable for further use as a raw material to produce a chemical.

From block 106, process 100 proceeds to block 108 where the purified biologically derived intermediate product from block 104 is used as a raw material for production of a chemical. In some embodiments, the purified biologically derived intermediate product may be subjected to pre-processing such as chemical digestion, denaturing, hydrogenation, decarboxylation. In embodiments, the biologically derived intermediate product is chemically digested into a purified biologically derived intermediate product with different functional groups, hydrocarbon chain lengths may be reduced, triacylglycerols may be hydrolyzed to form corresponding fatty acids. After any pre-processing steps, the purified biologically derived intermediate product may be chemically reacted with reactants to form a chemical. The reactions may be of any suitable type, including, but not limited to condensation reaction with and without condensate removal, free radical polymerization, esterification, alkoxylation, phosphonation, phosphorylation, sulfonation, quaternization, ring closing condensation, and combinations thereof. The selected reactants and type of reactions determine the chemical product. Some chemicals may include, but are not limited to, wetting agents, corrosion inhibitors, water clarifiers, stimulation chemicals, sand consolidation chemicals, salt control chemicals, ionic liquids, and petrochemical treatment chemicals. The produced chemical may be used “as is” or may be blended with a carrier fluid as well as additives, including, but not limited to, stabilizers, solubilizer, or any other chemicals required to make a finished product.

One specific method of producing a chemical may include producing a C₁₂-C₂₄ range fatty acid using a bacterial organism. The C₁₂-C₂₄ range fatty acid may be reacted with diethylene triamine in a condensation reaction to make an imidazoline corrosion inhibitor molecule.

Another method may include biologically digest a waste stream containing alkylbenzene to make alkylphenols. Thereafter the alkylphenols may be reacted with formaldehyde to form resins. The resins may be further alkoxylated to make Emulsion Breaker intermediates.

Another method may include use pulp and paper water waste streams to biologically produce starch. The starch may be digested to produce high molecular weight sugars across a molecular weight range. The high molecular weight sugars may be reacted with P2O5 to make phosphate esters, and with P2S5 to make thiophosphate esters.

FIG. 2 is a schematic illustration of a bioreactor system 200 in accordance with some embodiments of the present disclosure. Bioreactor system 200 comprises first bioreactor 202 and second bioreactor 204. First bioreactor 202 and second bioreactor 204 are illustrated as agitated batch mixers where first bioreactor 202 contains stirring implement 206 and second bioreactor 204 contains stirring implement 208. Stirring implement 206 and/or 208 may include any suitable type of stirring including paddle, magnetic stirring, or pump around, for example. While illustrated as stirred reactors, first bioreactor 202 and second bioreactor 204 may include any other type of suitable reactors, including, but not limited to, batch bioreactors, continuously stirred bioreactors, bubble column bioreactors, airlift bioreactors, fluidized bed bioreactors, packed bed bioreactors, photo bioreactors, and/or membrane bioreactors for example. Additionally, bioreactor system 200 illustrates two bioreactors in series, but there may be any number including three, four, five, or more bioreactors in series, parallel, or any combination thereof. Bioreactor system 200 can be configured to be utilized with any of the above referenced bacterial organisms and produce any desired biologically derived intermediate product 234.

First bioreactor 202 may have several inputs to support the bacterial organism. For example, first bioreactor 202 may include a source of nitrogen and phosphorus 210, a source of nutrient medium 212, a source of oxygen 214, and a source of pH control agent 216. Additionally, a hydrocarbon containing waste source 218 may be provided which contains a hydrocarbon the bacterial organism is capable of metabolizing to the desired biologically derived intermediate product 234. In embodiments, the hydrocarbon containing waste source 218 may include wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, and mine waste from tar sands mining as well as produced hydrocarbons such as crude oil, gas, and asphaltenes. First bioreactor 202 may include pH probe 222 and dissolved oxygen probe 224 which may provide signals to a control system to adjust pH and/or oxygen, such as by adjusting a flow of the source of pH control agent 216 and/or oxygen 214.

In some embodiments, first bioreactor 202 is loaded with a hydrocarbon containing waste from the hydrocarbon containing waste source 218 and one or more of a bacterial organism targeted for the hydrocarbon in the hydrocarbon containing waste 218 to produce an intermediate of the desired biologically derived intermediate product for second bioreactor 204. Then, the intermediate of the desired biologically derived intermediate product of first bioreactor 202 is transferred through valve 220 into second bioreactor 204 to be digested by the same or different bacterial organisms or combination thereof to produce the desired biologically derived intermediate product 234. Valve 220 may include any suitable type of valve that can regulate the flow of fluids and isolate bioreactors 202 and 204 such as butterfly valve, ball valve, gate valve, globe valve, check valve, plug valve, and actuated valve. Valve 220 may be connected to a control system to open or close or adjust the degree of opening to control the flow from first bioreactor 202 to second bioreactor 204.

In some embodiments, first bioreactor 202 and second bioreactor 202 may be maintained in different conditions such as different concentration of oxygen, nutrient medium, concentrations of nitrogen and phosphorous, pH, temperature, and pressure to optimize the digestion of the respective bacterial organisms. For instance, first bioreactor 202 may be maintained in specific aerobic conditions to optimize the digestion of the bacterial organisms selected in first bioreactor 202 to produce an intermediate of the desired biologically derived intermediate product for second bioreactor 204 through the control system that adjust the flow of oxygen from the source of oxygen 214 depending upon the reading of dissolved oxygen probe 224. In contrast, second bioreactor 204 may be maintained in anaerobic conditions or a concentration of oxygen different than in first bioreactor 202 through the control system that adjust the flow of oxygen from the source of oxygen 232 depending upon the reading of dissolved oxygen probe 228 to optimize the production of the desired biologically derived intermediate product 234. Further, first bioreactor 202 may be maintained at a pH to optimize the digestion of the bacterial organisms selected in first bioreactor 202 to produce an intermediate of the desired biologically derived intermediate product for second bioreactor 204 through the control system that adjusts the flow of the source of pH control agent 216 depending upon the reading of pH probe 222. In contrast, second bioreactor 204 may be maintained at a pH different than in first bioreactor 202 through the control system that adjusts the flow of the source of pH control agent 230 depending upon the reading of pH probe 226 to optimize the production the desired biologically derived intermediate product 234.

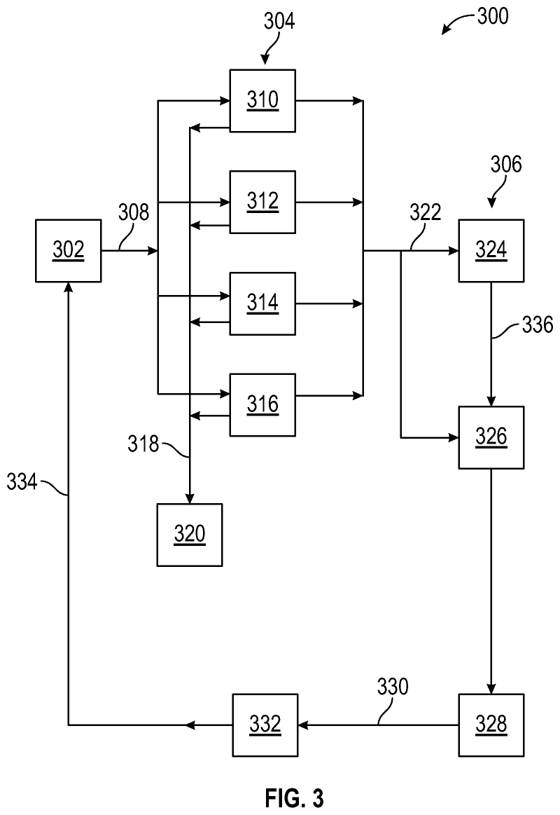

FIG. 3 is a schematic illustration of an integrated refinery chemical production system 300 for producing chemicals from a hydrocarbon containing waste source in accordance with some embodiments of the present disclosure. Integrated refinery chemical system 300 may integrate steps of chemical production including souring of precursor molecules, metabolism of the precursor molecules to a biologically derived intermediate product and using the biologically derived intermediate product to produce the chemical. As illustrated in FIG. 3, integrated refinery chemical production system 300 may include a hydrocarbon containing waste source 302, a bioreactor bank 304, and chemical plant 306.

Hydrocarbon containing waste source 302 may include any of the previously discussed hydrocarbon sources, including, industrial sources such as wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, and mine waste from tar sands mining as well as produced hydrocarbons such as crude oil, gas, and asphaltenes. While illustrated as one hydrocarbon containing waste source two or more hydrocarbon containing waste sources. Hydrocarbon containing waste from hydrocarbon containing waste source 302 may be introduced into the bioreactor bank 304 through line 308. The hydrocarbon containing waste may be used “as is” from the hydrocarbon containing waste source 302 or may be treated prior to introduction into bioreactor bank 304. Treating may include any suitable treating process such as de-watering. While bioreactor bank 304 is illustrated as comprising four bioreactors in parallel (first bioreactor 310, second bioreactor 312, third bioreactor 314, and fourth bioreactor 316), any number of reactors may be utilized in series or parallel. The bioreactors may be of any type including, but not limited to, batch reactor, continuously stirred reactors, bubble column reactors, airlift reactors, fluidized bed reactors, packed bed reactors, photo reactors, and/or membrane reactors for example. Bioreactors in bioreactor bank 304 may utilize any of the bacterial organism described above to metabolize the hydrocarbon to produce a biologically derived intermediate product. In some embodiments, each bioreactor in the bioreactor bank 304 may produce the same biologically derived intermediate product. In further embodiments, the bioreactors may produce different biologically derived intermediate products. For example, fats may be produced in first bioreactor 310, fatty acids may be produced in second bioreactor 312, sugars may be produced in third bioreactor 314, and amines may be produced in fourth bioreactor 312. Treated wastewater stream 318 may be withdrawn from bioreactor bank 304 and sent to wastewater treatment plant 320 for further treatment.

The biologically derived intermediate products from each of the bioreactors in bioreactor bank 304 may be withdrawn from the bioreactors and introduced into chemical plant 306 through line 322. Optionally, the biologically derived intermediate products may be purified and/or processed further before introduction into chemical plant 306. For example, the biologically derived intermediate products may be introduced into a separation unit, which may include any suitable unit operations such as extraction, leaching, flotation, flocculation, filtration, fractional distillation, precipitation, recrystallization, stripping, chelation, filtration, centrifugation, or any combinations thereof, to separate at least a portion of the biologically derived intermediate product.

Chemical plant 306 may include chemical reactor 324 and a chemical blending unit 326, chemical reactor 324 may include any type of reactors including, but not limited to, batch reactor, continuously stirred reactors, bubble column reactors, airlift reactors, fluidized bed reactors, packed bed reactors, photo reactors, and/or membrane reactors for example. In embodiments, there may be multiple reactors in series or parallel, chemical reactor 324 may take as a feed the biologically derived intermediate product and react the biologically derived intermediate product to produce a chemical. In embodiments, the chemicals may include, but are not limited to, wetting agents, corrosion inhibitors, water clarifiers, stimulation chemicals, sand consolidation chemicals, salt control chemicals, ionic liquids, and petrochemical treatment chemicals, for example. The produced chemical from chemical reactor 324 may be used “as is” or may be routed to chemical blending unit 326 via line 336 to be blended with a carrier fluid as well as additives, including, but not limited to, stabilizers, solubilizer, or any other chemicals required to make a finished product.

The chemicals blending unit 326 may be any blending unit capable of combining chemicals with carrier fluids and/or additives to achieve a new combined product with its own unique properties. The chemicals blending unit 326 may be tailored for dry chemicals or gentle blending or liquid blending. The products of chemicals blending unit 326 may be used as raw materials in various consumer goods, such as lubricants, cosmetics, paints, perfumes, candles, sealants, inks, and polishes.

The products of chemicals blending unit 326 may be routed to a refinery 328. The chemicals may be used in units within the refinery 328 to exert beneficial effects to the units. A refinery wastewater stream 330 may be withdrawn from refinery 328 and be routed to refinery wastewater treatment unit 332. Refinery wastewater may contain hydrocarbons which may be metabolized to form biologically derived intermediate products. In embodiments, refinery wastewater from refinery wastewater treatment unit 332 may be routed to hydrocarbon containing waste source 302. Refinery wastewater treatment unit 332 may be any wastewater treatment that removes and eliminates contaminants from wastewater and converts it into an effluent that can be returned to the water cycle. Once returned to the water cycle, the effluent creates an acceptable impact on the environment or is reused for various purposes. The refinery wastewater treatment plant 332 includes, but is not limited to, brine treatment, solids removal through filtration or chemical precipitation for instance, oils and grease removal, removal of biodegradable organics, removal of other organics, removal of acids and alkalis, and removal of toxic materials. The refinery may include a petrochemical refinery. Petrochemical includes, but is not limited to, all the aliphatic, aromatic, and naphthenic organic chemicals, as well as carbon black and such inorganic materials as sulfur and ammonia. Products made from petrochemicals include plastics, soaps and detergents, solvents, drugs, fertilizers, pesticides, explosives, synthetic fibres and rubbers, paints, epoxy resins, and flooring and insulating materials. Petrochemicals are found in products as diverse as aspirin, luggage, boats, automobiles, aircraft, polyester clothes, and recording discs and tapes. Refinery 328 may be any industrial process plant where crude oil is transformed and refined into useful products such as gasoline, diesel fuel, asphalt base, fuel oils, heating oil, kerosene, liquefied petroleum gas and petroleum naphtha. Refinery 328 comprises a number of different processing units including, but not limited to, separation unit, conversion unit, treating unit, and supporting process units. Supporting process units include, but is not limited to, crude desalting, atmospheric distillation, vacuum distillation, coker, visbreaker, resid hydrocracker, catalytic cracking process such as fluid catalytic cracking, de-asphalting, solvent extraction, hydrocracker, reformer, alkylation, polymerization, isomerization, hydrogen production, hydrogen purification, hydrotreater, BTX plant, for example.

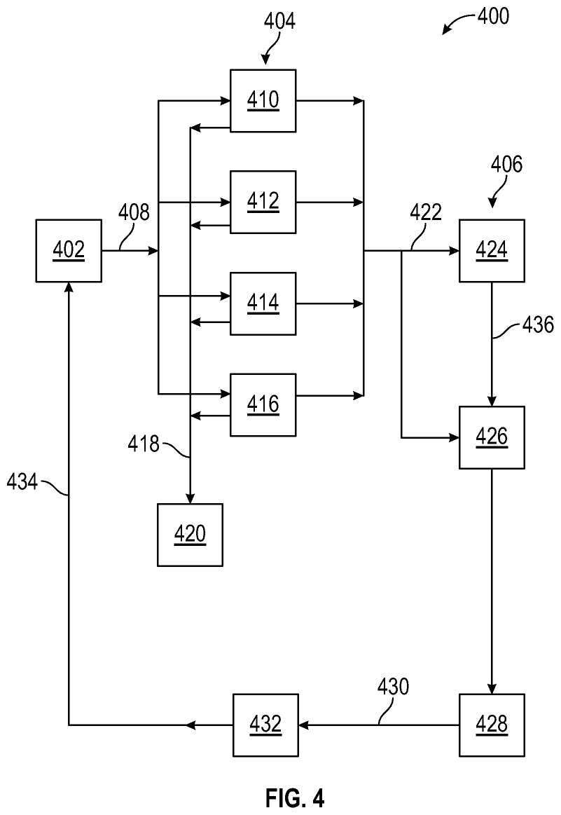

FIG. 4 is a schematic illustration of an integrated oilfield chemical production system 400 for producing chemicals from a hydrocarbon containing waste source in accordance with some embodiments of the present disclosure. Integrated oilfield chemical system 400 may integrate steps of oilfield chemical production including souring of precursor molecules, metabolism of the precursor molecules to a biologically derived intermediate product and using the biologically derived intermediate product to produce the oilfield chemical. As illustrated in FIG. 4, integrated oilfield chemical production system 400 may include a hydrocarbon containing waste source 402, a bioreactor bank 404, and chemical plant 406.

Hydrocarbon containing waste source 402 may include any of the previously discussed hydrocarbon sources, including, industrial sources such as wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, and mine waste from tar sands mining as well as produced hydrocarbons such as crude oil, gas, and asphaltenes. While illustrated as one hydrocarbon containing waste source two or more hydrocarbon containing waste sources. Hydrocarbon containing waste from hydrocarbon containing waste source 402 may be introduced into the bioreactor bank 404 through line 408. The hydrocarbon containing waste may be used “as is” from the hydrocarbon containing waste source 402 or may be treated prior to introduction into bioreactor bank 404. Treating may include any suitable treating process such as de-watering. While bioreactor bank 404 is illustrated as comprising four bioreactors in parallel (first bioreactor 410, second bioreactor 412, third bioreactor 414, and fourth bioreactor 416), any number of reactors may be utilized in series or parallel. The bioreactors may be of any type including, but not limited to, batch reactor, continuously stirred reactors, bubble column reactors, airlift reactors, fluidized bed reactors, packed bed reactors, photo reactors, and/or membrane reactors for example. Bioreactors in bioreactor bank 404 may utilize any of the bacterial organism described above to metabolize the hydrocarbon to produce a biologically derived intermediate product. In some embodiments, each bioreactor in the bioreactor bank 404 may produce the same biologically derived intermediate product. In further embodiments, the bioreactors may produce different biologically derived intermediate products. For example, fats may be produced in first bioreactor 410, fatty acids may be produced in second bioreactor 412, sugars may be produced in third bioreactor 414, and amines may be produced in fourth bioreactor 412. Treated wastewater stream 418 may be withdrawn from bioreactor bank 404 and sent to wastewater treatment plant 420 for further treatment.

The biologically derived intermediate products from each of the bioreactors in bioreactor bank 404 may be withdrawn from the bioreactors and introduced into chemical plant 406 through line 422. Optionally, the biologically derived intermediate products may be purified and/or processed further before introduction into chemical plant 406. For example, the biologically derived intermediate products may be introduced into a separation unit, which may include any suitable unit operations such as extraction, leaching, flotation, flocculation, filtration, fractional distillation, precipitation, recrystallization, stripping, chelation, filtration, centrifugation, or any combinations thereof, to separate at least a portion of the biologically derived intermediate product.

Chemical plant 406 may include oilfield chemical reactor 424 and an oilfield chemical blending unit 426. Oilfield chemical reactor 424 may include any type of reactors including, but not limited to, batch reactor, continuously stirred reactors, bubble column reactors, airlift reactors, fluidized bed reactors, packed bed reactors, photo reactors, and/or membrane reactors for example. In embodiments, there may be multiple reactors in series or parallel. Oilfield chemical reactor 424 may take as a feed the biologically derived intermediate product and react the biologically derived intermediate product to produce an oilfield chemical. In embodiments. the oilfield chemicals may include, but are not limited to, wetting agents, corrosion inhibitors, water clarifiers, stimulation chemicals, sand consolidation chemicals, salt control chemicals, ionic liquids, and petrochemical treatment chemicals, for example. The produced chemical from chemical reactor 424 may be used “as is” or may be routed to chemical blending unit 426 via line 436 to be blended with a carrier fluid as well as additives, including, but not limited to, stabilizers, solubilizer, or any other chemicals required to make a finished product.

The oilfield chemicals blending unit 426 may be any blending unit capable of combining chemicals with carrier fluids and/or additives to achieve a new combined product with its own unique properties. The oilfield chemicals blending unit 426 may be tailored for dry chemicals or gentle blending or liquid blending. The products of oilfield chemicals blending unit 426 may be used in oilfield applications.

The products of oilfield chemicals blending unit 426 may be routed to an oilfield location 428. The oilfield chemicals may be used in applications at the oilfield location 428 such as in drilling, cementing, production, overhaul, logging, exploration, completions, well construction, and abandonment, for example. An oilfield wastewater stream 430 may be withdrawn from oilfield location 428 and be routed to oilfield wastewater treatment 432. Oilfield wastewater may contain hydrocarbons which may be metabolized to form biologically derived intermediate products. In embodiments, oilfield wastewater from oilfield wastewater treatment unit 432 may be routed to hydrocarbon containing waste source 402.

Accordingly, the present disclosure may generally relate to production of chemicals. The following statements may describe certain embodiments of the disclosure but should be read to be limiting to any particular embodiment.

Statement 1. A method comprising: introducing a hydrocarbon containing waste source into a bioreactor; metabolizing at least a portion of hydrocarbons from the hydrocarbon containing waste source using a bacterial organism to form a biologically derived intermediate product; and introducing at least a portion of the biologically derived intermediate product into a chemical reactor and reacting at least a portion of the biologically derived intermediate product to form a chemical product.

Statement 2. The method of statement I wherein the hydrocarbon containing waste source comprises at least one source selected from the group consisting of wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, mine waste from tar sands mining, and combinations thereof.

Statement 3. The method of statement 2 further comprising dewatering the hydrocarbon containing waste source before introducing the hydrocarbon containing waste source into the bioreactor.

Statement 4. The method of any of statements 1-3 wherein the bioreactor comprises at least one reactor selected from the group consisting of continuously stirred bioreactors, bubble column bioreactors, airlift bioreactors, fluidized bed bioreactors, packed bed bioreactors, photo bioreactors, membrane bioreactors, and combinations thereof.

Statement 5. The method of any of statements 1-4 wherein the bioreactor comprises two or more bioreactors in series and/or parallel.

Statement 6. The method of any of statements 1-5 wherein the bacterial organism comprises at least one organism selected from the group consisting of Acidobacteria bacterium, Acidothermus cellulolyticus, Acinetobacter baumannii, Acinetobacter baylyi, Rhodoccus opacus, Acinetobacter baumannii, Aeromonas hydrophila, Aeromonas salmonicida, Alcaligenes europhus, Alcanivorax borkumensis, Alcanivorax jadensis, Alteromonas macleodii, Anaeromyxobacter dehalogenans, Anaeromyxobacter, Rhodoccus erythroplis, Arabidopsis thaliana, Bradyrhizobium japonicum, Cryptococcus curvatus, Erythrobacter litoralis, Fundibacter jadensis, gamma proteobacterium, Hahella chejuensis, Rhodoccus fascians, marine gamma proteobacterium, Marinobacter algicola, Marinobacter aquaeolei, Marinobacter hydrocarbinoclasticus, Rhodoccus ruber, Rhodoccus jostii; Nocardia asteroides, Nocardia corallina, Nocardia globerula, Nocardia resticta; Methylibium petroleiphilum, Microscilla marina, Mortierella alpina, Mus musculus, Mycobacterium abscessus, Mycobacterium avium, Mycobacterium avium, Mycobacterium bovis, Mycobacterium gilvum, Mycobacterium leprae, Mycobacterium marinum, Mycobacterium smegmatis, Mycobacterium tuberculosis, Mycobaterium smegmatis, Mycobacterium ratisbonense; Mycobacterium ulcerans, Mycobacterium vanbaalenii, Myxococcus xanthus, Natronomonas pharaonis, Nocardia farcinica, Dietzia maris; Gordonia amarae, Streptomyces coelicolor, Photobacterium profundum, Plesiocystis pacifica, Polaromonas naphthalenivorans, Psudomonas aeruginosa, Psychrobacter arcticus, Psychrobacter cryohalolentis, Rhodococcus opacus, Rhodoferax ferrireducens, Rhodoferax ferrireducens, Roseiflexus sp., Roseiflexus castenholzii, Saccharomyces cerevisiae, Saccharopolyspora erythraea, Salinibacter ruber, Simmodsia chinensis, Solibacter usitatus, Sphingopyxis alaskensis, Stigmatella aurantiaca, Streptomyces avermitilis, Streptomyces coelicolor, Streptomyces griseus, Ustilago maydis, Desulfovibrio, Pseudomonas, Azotobacter, Escheri coli, and combinations thereof.

Statement 7. The method of any of statements 1-6 wherein the biologically derived intermediate product comprises at least one product selected from the group consisting of C1-C30 alcohols, C1-C30 hydrocarbons, C1-C30 alkanes, C1-C30 alkenes, C1-C30 alkynes, C6-C30 aromatics, C2-C30 acetates, C2-C30 alkaline earth acetates, carboxylic acids such as acetic acid, or in general, C2-C30 carboxylic acids, C2-C30 olefins, syngas, C2-C30 wax esters, triacylglycerols with C2-C30 fatty acid side chains, C2-C30 aldehydes, C2-C30 ketones, C2-C30 acid anhydrides, C2-C30 acyl halides, C2-C30 ethers, C2-C30 epoxies, C2-C30 mono and higher order amines, C2-C30 amides, C2-C30 enamines, C2-C30 ketals, C2-C30 lactones, C2-C30 nitrates, C2-C30 nitrites, C2-C30 nitriles, C2-C30 nitro containing compounds, C2-C30 alkanolamides, C2-C30 amidoamines, C2-C30 pyrenes, C2-C30 nitroso containing compounds, C2-C30 imines, C2-C30 imides, C2-C30 azides, C2-C30 cyanates, C2-C30 isocyanates, C2-C30 azo compounds, C2-C30 thiols, C2-C30 sulfides, C2-C30 disulfides, C2-C30 sulfoxides, C2-C30 sulfones, C2-C30 sulfinic acids, C2-C30 sulfonic acids, C2-C30 sulfonate esters, C2-C30 tyiocyanate, C2-C30 iscothiocyanate, C2-C30 thials, C2-C30 thioketones, and combinations thereof.

Statement 8. The method of any of statements 1-7 wherein the biologically derived intermediate product introduced into the chemical reactor has a biogenic carbon content of greater than 50 wt. % as measured by ASTM D6866-22.

Statement 9. The method of any of statements 1-8 wherein the chemical reactor comprises at least one reactor selected from the group consisting of continuously stirred reactors, bubble column reactors, airlift reactors, fluidized bed reactors, packed bed reactors, photo reactors, membrane reactors, and combinations thereof.

Statement 10. The method of any of statements 1-9 wherein the chemical reactor comprises two or more bioreactors in series and/or parallel.

Statement 11. The method of any of statements 1-10 wherein the chemical product comprises at least one chemical selected from the group consisting of wetting agents, corrosion inhibitors, water clarifiers, stimulation chemicals, sand consolidation chemicals, salt control chemicals, ionic liquids, petrochemical treatment chemicals, and combinations thereof.

Statement 12. The method of any of statements 1-11 wherein the biologically derived intermediate product is further introduced into a separation unit before introduction into the chemical reactor wherein the separation unit is configured to purify the biologically derived intermediate product.

Statement 13. The method of statement 12 wherein the separation unit utilizes at least one of extraction, leaching, flotation, flocculation, filtration, fractional distillation, precipitation, recrystallization, stripping, chelation, filtration, centrifugation, or any combinations thereof to purify the biologically derived intermediate product.

Statement 14. The method of any of statements 1-13 further comprising blending the chemical product with at least one of a carrier fluid, stabilizers, solubilizer, to make a finished product.

Statement 15. The method of any of statements 1-14 further comprising introducing the chemical product into a refinery.

Statement 16. The method of any of statements 1-5 further comprising applying the chemical product in an oilfield application.

Statement 17. A method comprising: introducing a hydrocarbon containing waste source into a bioreactor, wherein the hydrocarbon containing waste source comprises produced water from an oilwell; metabolizing at least a portion of hydrocarbons from the hydrocarbon containing waste source using a bacterial organism to form a biologically derived intermediate product; introducing at least a portion of the biologically derived intermediate product into a chemical reactor and reacting at least a portion of the biologically derived intermediate product to form an oilfield chemical product; and applying the oilfield chemical product in an oilfield application.

Statement 18. The method of statement 17 wherein the oilfield chemical product comprises at least one chemical selected from the group consisting of wetting agents, corrosion inhibitors, water clarifiers, stimulation chemicals, sand consolidation chemicals, salt control chemicals, ionic liquids, petrochemical treatment chemicals, and combinations thereof.

Statement 19. The method of statement 18 further comprising blending the chemical product with at least one of a carrier fluid, stabilizers, solubilizer, to make a finished product and applying the finished product in an oilfield application.

Statement 20. The method of statement 18 wherein the hydrocarbon containing waste source further comprises at least one source selected from the group consisting of wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, mine waste from tar sands mining, and combinations thereof.

It should be understood that the compositions and methods are described in terms of “comprising.” “containing.” or “including” various components or steps, the compositions and methods can also “consist essentially of” or “consist of” the various components and steps. Moreover, the indefinite articles “a” or “an,” as used in the claims, are defined herein to mean one or more than one of the elements that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular examples disclosed above are illustrative only, as the present disclosure may be modified and practiced in different but equivalent manners apparent to those skilled in the art having the benefit of the teachings herein. Although individual examples are discussed, the disclosure covers all combinations of all those examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

What is claimed is:

1. A method comprising:
collecting produced water comprising produced hydrocarbon comprising crude oil, gas, asphaltene, or any combinations thereof from at least one oil and gas well;
dewatering the produced water comprising produced hydrocarbon;
stripping contaminants from the dewatered produced hydrocarbon;
introducing the dewatered produced hydrocarbon into a bioreactor system;
metabolizing at least a portion of hydrocarbons from the produced hydrocarbon using a bacterial organism to form a biologically derived intermediate product;
introducing at least a portion of the biologically derived intermediate product into a chemical reactor system and reacting at least a portion of the biologically derived intermediate product to form a chemical product; and
applying the chemical product in an oilfield application, wherein the oilfield application comprises at least one oilfield application selected from the group consisting of drilling, cementing, production, overhaul, logging, exploration, completions, well construction, abandonment, and any combinations thereof.

2. (canceled)

3. (canceled)

4. The method of claim 1 wherein the bioreactor system comprises at least one bioreactor selected from the group consisting of continuously stirred bioreactors, bubble column bioreactors, airlift bioreactors, fluidized bed bioreactors, packed bed bioreactors, photo bioreactors, membrane bioreactors, and combinations thereof.

5. The method of claim 1 wherein the bioreactor system comprises two or more bioreactors in series and/or parallel.

6. The method of claim 1 wherein the bacterial organism comprises at least one organism selected from the group consisting of Acidobacteria bacterium, Acidothermus cellulolyticus, Acinetobacter baumannii, Acinetobacter baylyi, Rhodococcus opacus, Acinetobacter baumannii, Aeromonas hydrophila, Aeromonas salmonicida, Alcaligenes europhus, Alcanivorax borkumensis, Alcanivorax jadensis, Alteromonas macleodii, Anaeromyxobacter dehalogenans, Anaeromyxobacter, Rhodococcus erythroplis, Bradyrhizobium japonicum, Cryptococcus curvatus, Erythrobacter litoralis, Fundibacter jadensis, gamma proteobacterium, Hahella chejuensis, Rhodococcus fascians, marine gamma proteobacterium, Marinobacter algicola, Marinobacter aquaeolei, Marinobacter hydrocarbinoclasticus, Rhodococcus ruber, Rhodococcus jostii; Nocardia asteroides, Nocardia corallina, Nocardia globerula, Nocardia resticta; Methylibium petroleiphilum, Microscilla marina, Mortierella alpina, Mycobacterium abscessus, Mycobacterium avium, Mycobacterium avium, Mycobacterium bovis, Mycobacterium gilvum, Mycobacterium leprae, Mycobacterium marinum, Mycobacterium smegmatis, Mycobacterium tuberculosis, Mycobaterium smegmatis, Mycobacterium ratisbonense; Mycobacterium ulcerans, Mycobacterium vanbaalenii, Myxococcus xanthus, Natronomonas pharaonis, Nocardia farcinica, Dietzia maris; Gordonia amarae, Streptomyces coelicolor, Photobacterium profundum, Plesiocystis pacifica, Polaromonas naphthalenivorans, Psudomonas aeruginosa, Psychrobacter arcticus, Psychrobacter cryohalolentis, Rhodococcus opacus, Rhodoferax ferrireducens, Rhodoferax ferrireducens, Roseiflexus sp., Roseiflexus castenholzii, Saccharomyces cerevisiae, Saccharopolyspora erythraea, Salinibacter ruber, Simmodsia chinensis, Solibacter usitatus, Sphingopyxis alaskensis, Stigmatella aurantiaca, Streptomyces avermitilis, Streptomyces coelicolor, Streptomyces griseus, Ustilago maydis, Desulfovibrio, Pseudomonas, Azotobacter, Escherichia coli, and combinations thereof.

7. The method of claim 1 wherein the biologically derived intermediate product comprises at least one product selected from the group consisting of C1-C30 alcohols, C1-C30 hydrocarbons, C1-C30 alkanes, C1-C30 alkenes, C1-C30 alkynes, C6-C30 aromatics, C2-C30 acetates, C2-C30 alkaline earth acetates, carboxylic acids such as acetic acid, or in general, C2-C30 carboxylic acids, C2-C30 olefins, syngas, C2-C30 wax esters, triacylglycerols with C2-C30 fatty acid side chains, C2-C30 aldehydes, C2-C30 ketones, C2-C30 acid anhydrides, C2-C30 acyl halides, C2-C30 ethers, C2-C30 epoxies, C2-C30 mono and higher order amines, C2-C30 amides, C2-C30 enamines, C2-C30 ketals, C2-C30 lactones, C2-C30 nitrates, C2-C30 nitrites, C2-C30 nitriles, C2-C30 nitro containing compounds, C2-C30 alkanolamides, C2-C30 amidoamines, C2-C30 pyrenes, C2-C30 nitroso containing compounds, C2-C30 imines, C2-C30 imides, C2-C30 azides, C2-C30 cyanates, C2-C30 isocyanates, C2-C30 azo compounds, C2-C30 thiols, C2-C30 sulfides, C2-C30 disulfides, C2-C30 sulfoxides, C2-C30 sulfones, C2-C30 sulfinic acids, C2-C30 sulfonic acids, C2-C30 sulfonate esters, C2-C30 tyiocyanate, C2-C30 iscothiocyanate, C2-C30 thials, C2-C30 thioketones, and combinations thereof.

8. The method of claim 1, wherein the biologically derived intermediate product introduced into the chemical reactor system has a biogenic carbon content of greater than 50 wt. % as measured by ASTM D6866-22.

9. The method of claim 1, wherein the chemical reactor system comprises at least one reactor selected from the group consisting of continuously stirred reactors, bubble column reactors, airlift reactors, fluidized bed reactors, packed bed reactors, photo reactors, membrane reactors, and combinations thereof.

10. The method of claim 1, wherein the chemical reactor comprises two or more bioreactors in series and/or parallel.

11. The method of claim 1 wherein the chemical product comprises at least one chemical selected from the group consisting of wetting agents, corrosion inhibitors, water clarifiers, stimulation chemicals, sand consolidation chemicals, salt control chemicals, ionic liquids, petrochemical treatment chemicals, and combinations thereof.

12. The method of claim 1 wherein the biologically derived intermediate product is further introduced into a separation unit before introduction into the chemical reactor wherein the separation unit is configured to purify the biologically derived intermediate product.

13. The method of claim 12 wherein the separation unit utilizes at least one of extraction, leaching, flotation, flocculation, filtration, fractional distillation, precipitation, recrystallization, stripping, chelation, filtration, centrifugation, or any combinations thereof to purify the biologically derived intermediate product.

14. The method of claim 1 further comprising blending the chemical product with at least one of a carrier fluid, stabilizers, solubilizer, to make a finished product.

15. The method of claim 1 further comprising introducing the chemical product into a refinery.

16. (canceled)

17. A method comprising:
collecting produced water comprising produced hydrocarbon comprising crude oil, gas, asphaltene, or any combinations thereof from at least one oil and gas well;
dewatering the produced water comprising produced hydrocarbon;
stripping contaminants from the dewatered produced hydrocarbon;
introducing the dewatered produced hydrocarbon into a bioreactor system;
metabolizing at least a portion of hydrocarbons from the produced hydrocarbon using a bacterial organism to form a biologically derived intermediate product;
introducing at least a portion of the biologically derived intermediate product into a chemical reactor and reacting at least a portion of the biologically derived intermediate product to form an oilfield chemical product; and
applying the oilfield chemical product in an oilfield application, wherein the oilfield application comprises at least one oilfield application selected from the group consisting of drilling, cementing, production, overhaul, logging, exploration, completions, well construction, abandonment, and any combinations thereof.

18. The method of claim 17 wherein the oilfield chemical product comprises at least one chemical selected from the group consisting of wetting agents, corrosion inhibitors, water clarifiers, stimulation chemicals, sand consolidation chemicals, salt control chemicals, ionic liquids, petrochemical treatment chemicals, and combinations thereof.

19. The method of claim 17, further comprising blending the chemical product with at least one of a carrier fluid, stabilizers, solubilizer, to make a finished product and applying the finished product in an oilfield application.

20. The method of claim 17, wherein the hydrocarbon containing waste source further comprises at least one source selected from the group consisting of wastewater skims, produced water from oilfield applications, gas plant debutanizer bottoms, palm oil mill effluent, waste streams from petrochemical plants, waste bottoms from crude oil processing plants, pulp and paper waste streams, waste streams from petroleum refining, mine waste from tar sands mining, and combinations thereof.
